(19) United States
(12) Patent Application Publication
(10) Pub. No.: US 2005/0216200 A1
(43) Pub. Date: Sep. 29, 2005

(54) Neural network pattern recognition for predicting pharmacodynamics using patient characteristics

(75) Inventors: Mirna Urquidi-MacDonald, State College, PA (US); Darrell Abernethy, Annapolis, MD (US)

Correspondence Address: LEYDIG, VOIT & MAYER, LTD., 700 THIRTEENTH STREET, N.W., SUITE 300, WASHINGTON, DC, 20005-3960, US

(73) Assignees: The Govt. of U.S.A. Represented by the Secretary, Department of Health and Human Services, Rockville, MD (US); The Penn State Research Foundation, University Park, PA (US)

(21) Appl. No.: 10/810,809

(22) Filed: Mar. 29, 2004

(51) Int. Cl. G06F 17/60; G01N 33/48; G01N 33/50

(52) U.S. Cl. 702/19; 705/3

(57) ABSTRACT

Methods are provided for predicting the effect of a drug given the drug dose and individual patient clinical characteristics. A neural network is trained on samples of clinical data including the observed drug dose and effect on patients, as well as their individual clinical characteristics. The neural network is then validated to ensure that its predictions fall within an acceptable error range. The neural network is used to predict the effect of a given drug dose for a given set of individual patient clinical characteristics. Methods are also provided for predicting the drug dose required to achieve a desired effect. Another neural network is trained on samples of clinical data including the observed drug dose and effect on patients, as well as their individual clinical characteristics. The neural network is then validated to ensure that its predictions fall within an acceptable error range. The neural network is used to predict the dose of a drug dose required to achieve a desired effect for a patient with a given set of individual clinical characteristics. The first neural network is used to generate training data for the second neural network.

### FIELD OF THE INVENTION

This invention pertains to the prediction of drug dose for a desired drug effect, and drug effect for a given drug dose, and more particularly to the use of artificial neural networks to make those predictions in view of individual patient characteristics.

### BACKGROUND OF THE INVENTION

The term narrow therapeutic index (NTI), or narrow therapeutic ratio, has been used in the art to refer to drugs that have a narrow range between the dose needed for a beneficial effect and the dose causing a toxic effect. These drugs often require constant patient monitoring so that the level of medication can be adjusted as necessary to assure uniform and safe results. This monitoring is often achieved either by drug therapeutic concentration monitoring or pharmacodynamic monitoring. However, there are many circumstances when neither drug plasma concentration nor therapeutic effect is available in real time. The use of NTI drugs is further complicated by the variability of patient response to the drugs. For example, some patients may experience toxic serum concentrations close to that of the minimal therapeutic concentration. The sources of variability in therapeutic response to NTI drugs include the patient's clinical and personal characteristics, the process by which drug therapy is implemented and monitored, and lastly, the drug itself. Therefore, approaches to individualize patient treatment without concentration and effect data may provide an opportunity for improved use of some NTI drugs if dose predictions can be made within clinically acceptable variability.

Abciximab, the Fab fragment of the chimeric human murine monoclonal antibody 7E3, that binds to the glycoprotein (GP) IIb/IIIa receptor and inhibits platelet aggregation, is one drug with a narrow therapeutic index that has considerable inter-individual pharmacokinetic variability. Various efforts to monitor treatment with abciximab and other GP IIb/IIIa platelet receptor antagonists, including bleeding time, ex vivo inhibition of platelet aggregation, and receptor blockade have been evaluated and reviewed. Previous studies have shown that platelet activation may occur during acute coronary syndromes, and this is thought be, at least in part, related to the onset of thrombosis. Platelet activation results in exposure of the GP IIb/IIIa receptor, and abciximab occupation of the receptor may prevent it from binding fibrinogen and fibronectin, thereby preventing platelet bridging and platelet aggregate formation.

Abciximab is frequently administered during angioplasty procedures, with under-treatment possibly resulting in unsuccessful maintenance of arterial potency following angioplasty, and over-treatment possibly resulting in hemorrhage up to and including intracranial hemorrhage. Abciximab dose is weight corrected for the initial bolus dose, with a steady-state infusion following the bolus dose. The dose is based on data from large clinical trials that provided mean dose response data across the clinical trial population. There is wide inter-patient variability in both dose-response and concentration-response relationships. As neither abciximab concentration nor inhibition of platelet aggregation are likely to be available in real time for individualization of patient dose, there exists a need for methods to permit individualization of abciximab dose in a clinical setting.

Accordingly, there is a need to predict the effect of a dose of drugs that have a narrow therapeutic index or narrow therapeutic ratio (e.g., drugs such as abciximab, tissue plasminogen activator (TPA), cancer chemotherapy drugs such as cisplatin and doxorubicin, and arthritis treatment drugs such as tumor necrosis factor (TNF) alpha antibody) while accounting for individual patient characteristics. Likewise, there is a need to predict the dose of that drug needed to achieve a desired effect in an individual patient while accounting for that patient's characteristics.

### BRIEF SUMMARY OF THE INVENTION

The invention provides a method of predicting a drug dose necessary to achieve a desired drug effect using patient clinical characteristics. One embodiment of the invention includes the steps of inputting to a computer neural network a first data set comprising drug dose data, drug effect data, and patient characteristics data for a plurality of patients; training the computer neural network on the first data set; and using the computer neural network to predict a drug dose for a specific patient given a desired drug effect and patient characteristics of the specific patient. The computer neural network may be a backpropagation neural network using a steepest descent learning rule. The computer neural network is trained by establishing a relationship between the drug effect data and corresponding drug dose data and patient characteristics data.

In one embodiment of the invention, the computer neural network receives drug dose data and patient characteristics data, predicts a drug effect based on the drug dose data and the patient characteristics data, compares the predicted drug effect to received drug effect data, and adjusts a weight in the computer neural network based on a difference between the predicted drug effect and the received drug effect data. The computer neural network is validated using a second data set comprising drug dose data, drug effect data, and patient characteristics data for a plurality of patients. Validating includes inputting to the computer neural network the drug dose data and the patient characteristics data and comparing a predicted drug effect to the drug effect data corresponding to the inputted drug dose data and patient characteristics data.

In keeping with the features of the present invention, the drug dose data may be a drug dose versus time signature and the drug effect data may be a drug effect versus time signature. The patient characteristics data can include, but are not limited too, at least one of, and typically at least two of, data concerning ethnicity, age, gender, weight, stable angina, presence of diabetes, blood pressure, use of nitrates, cholesterol level, use of statins, use of beta blockers, use of calcium blockers, use of diuretics, smoking history, and history of previous myocardial infarctions. In one embodiment, the drug dose data concerns the drug abciximab and the drug effect data concerns the inhibition of adenosine diphosphate (ADP)-induced platelet aggregation. In this embodiment, patient characteristics data further include the use of other platelet aggregation inhibitors such as Ticlid and Clopid. Though no single input parameter controls a patient's response to abciximab, in an exemplary embodiment of the invention the patient characteristics data include at least weight, smoking history, and history of previous myocardial infarctions. In another exemplary embodiment of the invention, the patient characteristics include at least whether the patient has high levels of Ticlid or Clopid and has stable angina.

In other embodiments of the invention, drug dose data concerns one of other NTI drugs such as TPA, cisplatin, doxorubicin, and TNF alpha antibody. Drug effect data concerns data regarding the intended effect of the NTI drug.

Yet another embodiment of the invention relates to a method of predicting a drug dose necessary to achieve a desired drug effect using patient clinical characteristics. This method includes inputting to a first computer neural network a first data set comprising the drug dose data, drug effect data, and patient characteristics data for a plurality of patients; training the first computer neural network on the first data set; using the first computer neural network to generate a second data set comprising drug dose data, drug effect data, and patient characteristics data for a plurality of hypothetical patients; inputting to a second neural network the second data set; training the second neural network on the second data set; and using the second neural network to predict a drug dose for a specific patient given a desired drug effect and patient characteristics of the specific patient. In this embodiment, first computer neural network and the second computer neural network may be backpropagation neural networks using a steepest descent learning rule.

In one embodiment of the invention, training the first computer neural network comprises establishing a relationship between the drug effect data and corresponding drug dose data and patient characteristics data. The first computer neural network receives drug dose data and patient characteristics data, predicts a drug effect based on the drug dose data and the patient characteristics data, compares the predicted drug effect to received drug effect data, and adjusts a weight in the first computer neural network based on a difference between the predicted drug effect and the received drug effect data. Training the second computer neural network also comprises establishing a relationship between the drug dose data and corresponding drug effect data and patient characteristics data. The second computer neural network receives drug effect data and patient characteristics data, predicts a drug dose based on the drug effect data and the patient characteristics data, compares the predicted drug dose to received drug dose data, and adjusts a weight in the second computer neural network based on a difference between the predicted drug dose and the received drug dose data.

A further embodiment of the invention includes validating the first computer neural network includes using a third data set comprising drug dose data, drug effect data, and patient characteristics data for a plurality of patients. Validating the first computer neural network comprises inputting to the first computer neural network the drug dose data and the patient characteristics data, and comparing a predicted drug effect to the drug effect data corresponding to the inputted drug dose data and patient characteristics data. The embodiment also includes validating the second computer neural network using a third data set comprising drug dose data, drug effect data, and patient characteristics data for a plurality of patients. Validating the second computer neural network comprises inputting to the second computer neural network the drug effect data and the patient characteristics data, and comparing a predicted drug dose to the drug dose data corresponding to the inputted drug effect data and patient characteristics data.

Yet another embodiment of the invention includes training the second computer neural network on a fourth data set comprising drug dose data, drug effect data, and patient characteristics data for a plurality of patients. Furthermore, using the second neural network to predict a drug dose comprises inputting the desired drug effect data and the patient characteristics and obtaining a predicted drug dose from the neural network that achieves the desired drug effect for the specific patient.

A further embodiment of the invention relates to a computer-readable medium having thereon computer-readable instructions for executing the methods of the previous embodiments.

These and other advantages of the invention, as well as additional inventive features, will be apparent from the description of the invention provided herein.

### BRIEF DESCRIPTION OF THE DRAWINGS

FIG. 1 illustrates a conceptual diagram of an artificial neuron (node) in the neural network (NN);

FIG. 2 illustrates an exemplary NN;

FIG. 3 illustrates a conceptual diagram of the neural network effect predictor (NNEP);

FIG. 4 illustrates a flow diagram of the operation of the NNEP;

FIG. 5 illustrates a conceptual diagram of the neural network dose predictor (NNDP);

FIG. 6 illustrates a flow diagram of the operation of the NNDP;

FIG. 7 illustrates a graph of measured and NN-calculated % Baseline ADP (20 μM) Aggregation vs. Time for a first training data set;

FIG. 8 illustrates a graph of measured and NN-calculated % Baseline ADP (20 μM) Aggregation vs. Time for a second training data set;

FIG. 9 illustrates a graph of measured and NN-calculated % Baseline ADP (20 μM) Aggregation vs. Time for a never before seen data set;

FIG. 10 illustrates a graph of measured and NN-calculated % Baseline ADP (20 μM) Aggregation vs. Time for another never before seen data set;

FIG. 11 illustrates a graph of measured and NN-calculated % Baseline ADP (20 μM) Aggregation vs. Time for a first validating data set;

FIG. 12 illustrates a graph of measured and NN-calculated % Baseline ADP (20 μM) Aggregation vs. Time for a second validating data set;

FIG. 13 illustrates a graph of a desired % Baseline ADP (20 μM) Aggregation vs. Time signature;

FIG. 14 illustrates a graph of a NN-predicted and actually administered dose vs. time for a first actual patient;

FIG. 15 illustrates a graph of a NN-predicted and actually administered dose vs. time for a second actual patient;

FIG. 16 illustrates a graph of a NN-predicted and actually administered dose vs. time for a third actual patient;

FIG. 17 illustrates a graph of a NN-predicted dose vs. time for patients in Data Set No. 3 to maintain the desired % Baseline ADP (20 μM) Aggregation vs. Time signature of FIG. 13;

FIG. 18 illustrates a graph of a NN-predicted dose vs. time for patients in Data Set No. 2 to maintain the desired % Baseline ADP (20 μM) Aggregation vs. Time signature of FIG. 13;

FIG. 19 illustrates a graph of a NN-predicted dose vs. time for patients in Data Set No. 1 to maintain the desired % Baseline ADP (20 μM) Aggregation vs. Time signature of FIG. 13;

FIG. 20 illustrates a graph of another desired % Baseline ADP (20 μM) Aggregation vs. Time signature;

FIG. 21 illustrates a graph of a NN-predicted dose vs. time for patients in Data Set No. 3 to maintain the desired % Baseline ADP (20 μM) Aggregation vs. Time signature of FIG. 20;

FIG. 22 illustrates a graph of a NN-predicted dose vs. time for patients in Data Set No. 2 to maintain the desired % Baseline ADP (20 μM) Aggregation vs. Time signature of FIG. 20; and

FIG. 23 illustrates a graph of a NN-predicted dose vs. time for patients in Data Set No. 1 to maintain the desired % Baseline ADP (20 μM) Aggregation vs. Time signature of FIG. 20.

### DETAILED DESCRIPTION OF THE INVENTION

The invention relies on artificial neural networks to perform pattern recognition among data sets of drug dose, drug effect, and patient clinical characteristics. The neural network is trained to associate drug dose and patient characteristics with drug effect. Alternatively, the neural network is trained to associate a drug effect and patient characteristics with a drug dose. By establishing this associative mapping, the neural network can predict a drug effect for given drug doses and patient characteristics, as well as predict a drug dose for a given drug effect and patient characteristics. The associative mapping is established by setting and adjusting the weights of the connections between nodes in the neural network. The invention uses a feed-forward backpropagation neural network to model pharmacodynamic behavior and predict drug dosage. The mathematical principles underlying the neural network are described below.

Neural Networks

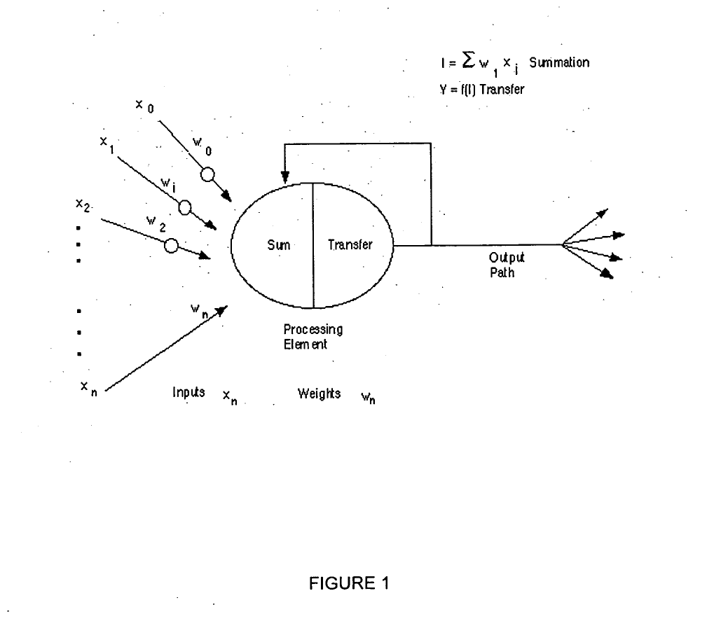

A mathematical representation of a single node is depicted in FIG. 1, which can also be considered as a simplified mathematical representation of a human neuron. A set of inputs (x₀ to xₙ), or input vector, X, is applied to a neuron. The input vector can be an external stimulus or outputs from another neuron. Each one of these inputs is multiplied by a corresponding weight (W₁ to Wₙ). The weighted inputs are then added together in a summation block. The weighted inputs are defined as a NET. The “nucleus” of the neuron then applies a transfer function to the imputing NET, as f(NET), and the value f(NET) becomes the output of that neuron.

Backpropagation

Backpropagation (BP) is a supervised, error-correcting learning algorithm. It realizes a gradient descent in error (“error” described as the difference of the actual output of the system and a target output).

A simpler version of backpropagation—delta rule on a perceptron—has been proven effective for finding solutions for all input-output mappings. The error surface in such networks has only one minimum, and the system moves on this error surface towards this minimum and remains there once it has reached it. A delta rule on a perceptron can be considered as a simplified case of backpropagation network. The error surface for the typical backpropagation net has local minima, and while searching for the solution the system can get “stuck” in a local error minimum. Modifications to the backpropagation exist to avoid this problem.

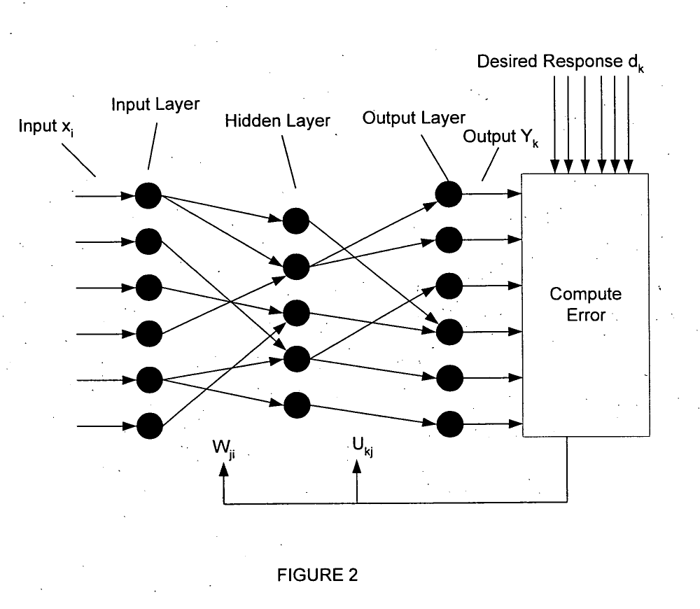

FIG. 2 illustrates a simplified representation of a 2-layer Back-Propagation (BP) NN. Yₖ indicates the BP NN output in neuron k (of the output layer) and dₖ the desired output associated to input xᵢ. Uₖⱼ and Wⱼᵢ are weight matrices representing the weighted connections between the input layer and the hidden layer and the hidden layer and the output layer, respectively. The weight matrices are adjusted as the error between Yₖ and dₖ is computed.

Characteristic of the BP NN is that the connectivity structure is feed-forward; that is, there are connections from the input layer nodes to the hidden layer nodes and from the hidden layer nodes to the output layer nodes, but there are no connections backward, for example, from the hidden layer nodes to the input layer nodes. There is also no lateral connectivity within the layers. Connectivity between the layers is complete in the sense that each input layer node is connected to each hidden layer node and each hidden layer node is connected to each output layer node. Weights connect the neurons between layers. Before learning, the weights of these connections are set to small random values. Backpropagation learning proceeds in the following way: an input pattern is chosen from a set of input patterns. This input pattern determines the activations of the input nodes. Setting the activations of the input layer nodes is followed by the activation forward propagation phase: the activation values of first the hidden units and then the output units are computed. This is done by using a transfer function such as the following:
hⱼ=1/(1+exp(−α(inputⱼ-θ)))=fⱼ(wⱼᵢ,xᵢ) (1)
where
[hⱼ]=activation of j^{th} node in a hidden layer
[Wⱼᵢ]=weight of connection from the j^{th} node in the hidden layer, to the i^{th} input node
[inputⱼ]=Σwⱼᵢ*xᵢ
[inputₖ]=Σuₖⱼ*hⱼ
[yₖ]=activation of k^{th} node in the output layer [=fₖ(uₖⱼ,hⱼ)]
[Uₖⱼ]=weight of connection from the k^{th} node in the output layer, to the j^{th} node in a hidden layer.
[α]=constant (determining the steepness of the sigmoid or transfer function)
[θ]=bias (determining the shift of the sigmoid function along the “input” axis).

Alternatively, the Tanh transfer function is used. It has outputs in the range −1 to 1 and can be written as:
hⱼ=2/(1+exp(−2*inputⱼ))−1 (2)

The derivative is: 1-hⱼ*hⱼ.

The bias is normally the part of the input coming from a “bias node”. The bias node has an activation of 1 during the whole learning process, is connected to each hidden and output layer node, and is fixed. However, bias connections are not necessary to solve non-linear separable problems when more than one layer is used. The weights of the bias connections are changed during the learning, just like all other weights.

The Learning Rule

The partial derivative of the error with respect to the output layer weights is:

$$\frac{\partial {E}_{x}}{\partial {u}_{kj}} = \frac{\partial {E}_{x}}{\partial {y}_{k}} \cdot \frac{\partial {y}_{k}}{\partial {u}_{kj}} \qquad ( 3 )$$

Equation (3) is obtained by multiplying the partial derivative of the error function, E[E= 1/2*Σ(dₖ−yₖ)²], by the derivative of the output generating function. If the error function equation 1/2*Σ(dₖ−yₖ)² is substituted into equation (3), the result is equations (4) to (6):

$$\begin{matrix}\frac{\partial {E}_{x}}{\partial {u}_{kj}} = \frac{\partial }{\partial {y}_{k}} [ \frac{1}{2} {\sum }_{a = 1}^{K} {( {d}_{a} - {y}_{a} )}^{2} ] \cdot \frac{\partial }{\partial {u}_{kj}} [ {f}_{k} ( {\sum }_{b = 0}^{M} {u}_{kb } \cdot {h}_{b} ) ] & ( 4 ) \\ \frac{\partial {E}_{x}}{\partial {u}_{kj}} = ( {y}_{k} - {d}_{k} ) \cdot {f}_{k}^{′} ( {\sum }_{b = 0}^{M} {u}_{kb } \cdot {h}_{b} ) \cdot {h}_{j} & ( 5 ) \\ \frac{\partial {E}_{x}}{\partial {u}_{kj}} = δ {y}_{k} \cdot {h}_{j} where & ( 6 ) \\ δ {y}_{k} = ( {y}_{k} - {d}_{k} ) \cdot {f}_{k}^{′} ( {\sum }_{b = 0}^{M} {u}_{kb } \cdot {h}_{b} ) & ( 7 )\end{matrix}$$

represents the backpropagating error related to the hidden layer (also called Δ).

The calculation of the change in error as a function of the hidden layer weights is more difficult because there is no way of getting “desired outputs” for the hidden layer neurons (or processing elements (PE)). It is only known what the network outputs should be. The partial derivative is similar to before but a little more complex:

$$\begin{matrix}\frac{\partial {E}_{x}}{\partial {w}_{ji}} = \frac{\partial }{\partial {w}_{ji}} [ \frac{1}{2} {\sum }_{a = 1}^{K} {( {d}_{a} - {y}_{a} )}^{2} ] & ( 8 ) \\ \frac{\partial {E}_{x}}{\partial {w}_{ji}} = {\sum }_{a = 1}^{K} \frac{\partial }{\partial {w}_{ji}} [ \frac{1}{2} {( {d}_{a} - {y}_{a} )}^{2} ] & ( 9 ) \\ \frac{\partial {E}_{x}}{\partial {w}_{ji}} = {\sum }_{a = 1}^{K} [ \frac{\partial }{\partial {y}_{a}} ( \frac{1}{2} {( {d}_{a} - {y}_{a} )}^{2} ) \cdot \frac{\partial {y}_{a}}{\partial {h}_{j}} \cdot \frac{\partial {h}_{j}}{\partial {w}_{ji}} ] & ( 10 ) \\ \frac{\partial {E}_{x}}{\partial {w}_{ji}} = [ {\sum }_{a = 1}^{K} ( {y}_{a} - {d}_{a} ) \cdot {f}_{k}^{′} ( {\sum }_{b = 0}^{M} {u}_{k} b \cdot {h}_{b} ) \cdot {u}_{aj} ] \cdot {f}_{j}^{′} ( {\sum }_{b = 0}^{p} {w}_{jb } \cdot {x}_{b} ) \cdot {x}_{i} & ( 11 ) \\ \frac{\partial {E}_{x}}{\partial {w}_{ji}} = δ {h}_{j} \cdot {x}_{i} where \text{:} & ( 12 ) \\ δ {h}_{j} = [ {\sum }_{a = 1}^{K} ( {y}_{a} - {d}_{a} ) \cdot {f}_{k}^{′} ( {\sum }_{b = 0}^{M} {u}_{kb} \cdot {h}_{b} ) \cdot {u}_{aj} ] \cdot {j}_{j}^{′} ( {\sum }_{b = 0}^{p} {w}_{jb } \cdot {x}_{b} ) & ( 13 )\end{matrix}$$

which represents the backpropagation of the error from the output layer to the hidden layer.

Weighting Error

In order to minimize the error all the weights should be adjusted in the opposite direction to the error gradient each time a training input/output vector pair is presented to the network as follows:

$$\begin{matrix}Δ {u}_{kj} = - η \cdot \frac{\partial {E}_{x}}{\partial {u}_{kj}} = - η \cdot δ {y}_{k} \cdot {h}_{j} & ( 14 ) \\ {u}_{kj}^{new} = {u}_{kj}^{old} + δ {u}_{kj} & ( 15 ) \\ δ {w}_{ji} = - μ \cdot \frac{\partial {E}_{x}}{\partial {w}_{ji}} = - μ \cdot δ {h}_{j} \cdot {x}_{i} & ( 16 ) \\ {w}_{ji}^{new} = {w}_{ji}^{old} + Δ {w}_{ji} & ( 17 )\end{matrix}$$

where μ and η are positive valued scalar gain or learning rate constants.

The learning rate is controlled by the scalar constants μ and η. These should be relatively small, i.e. μ and η<1. If they are too small the rate of convergence is slow, but if they are too large it may be difficult to converge once in the vicinity of a minimum since the estimate of the gradient is only valid locally. The ideal learning strategy may be to use relatively high values to start with and then reduce them as the training progresses. When there is only a finite training vector set, it is advantageous to continually select the individual training vector input/output pairs at random from the set rather than sequence through the set. The training may require hundreds of thousands or even millions of these iterations, especially for very complex problems.

The equations require an activation function which is differentiable, and if possible, one whose derivative is easy to compute. The sigmoid functions of equation (1) or (2) are a suitable choice of function because not only is it continuously and differentiable, but its derivative can be easily written as a function of the same original (not the derivative) function.

An Additional Momentum Factor

When the network weights approach a minimum solution, the gradient becomes small and the step size diminishes too, giving very slow convergence. If a so-called “momentum factor” is added to the weight update equations the weights can be updated with some component of past updates. This reduces the decay in learning updates and cause the learning to proceed through the weight space in a fairly constant direction. The benefits of this, in addition to faster convergence to the minimum, is that it may even be possible to escape a local minimum if there is enough momentum to travel through it and over the following hill.

Adding the momentum factor to the gradient descent learning equations (15) and
(17) Results in equations (18) and (19), respectively.
   W(k+1)=W(k)−μ∂Eₓ/∂W+α(W(k)−W(k−1)) (18)
   U(k+1)=U(k)−θ∂Eₓ/∂U+β(U(k)−U(k−1)) (19)
   where μ, η, α and β are positive valued scalar gain or learning rate constants, all less than 1. When the gradient has the same algebraic sign on consecutive iterations the weight change grows in magnitude. Thus momentum tends to accelerate descent in steady downhill directions. When the gradient has alternating algebraic signs on consecutive iterations the weight changes become smaller, thus stabilizing the learning by preventing oscillations.

Scaling Data

Scaling the data to train and test the networks is important in order to “assign” equivalent meaning to all vectors; i.e., if a vector varies from 10¹² to 10³⁴, and another varies from 10⁻⁶ to 10⁻², both should “contribute” to the learning equally. This is accomplished by scaling each input and output vectors to the same scale ([0,1] for a sigmoidal transfer function and [−1,1] for a bipolar-sigmoidal or hyperbolic tangent transfer function).

Fast-BP

When a Δ-rule (or any rule that is Δ-based) is adopted into the traditional BP NN, it is assumed that for a given input vector Xₘ={X_{m,1},X_{m,2}, . . . X_{m,P}} (bold indicates vectors, where P is the maximum anticipated number of variables, and m is the index for the number of training samples) the signal arriving to the neurons k of the (S=1) hidden layer is a linear weighted combination of the input vector

$${input}_{m , j} = {\sum }_{i = 1}^{P} {W}_{j , i , m} {x}_{m , i} \qquad ( 20 )$$

and the output of that neuron is given by
   h_{m,j}=f(input_{m,j}) (21)
where f( ) is a transfer function.

When a Δ-rule is used, the derivative of h_{m,j}, with respect to the weight vector W_{j,m}, is assumed to be

$$\begin{matrix}\frac{\partial {h}_{m , j}}{\partial {W}_{j , m}} = \frac{\partial f ( {input}_{m , j} )}{\partial {input}_{m , j}} \frac{\partial {input}_{m , j}}{\partial {W}_{j , m}} , and & ( 22 ) \\ \frac{\partial {input}_{m , j}}{\partial {W}_{j , m}} = {X}_{m} & ( 23 )\end{matrix}$$

This is the traditional way used in the derivation of the Δ-rule.

To train a supervised net a set of input and output variables are established, and several examples, shown as input/output pairs, are provided. In these examples vector Xₘ is the m^{th} input sample of the matrix X (of size P×M), and the dimension of Xₘ is P (P input variables). Each element of Xₘ can be noted xₘᵢ, with i=1,2 . . . P, and m=1,2, . . . M. M represents the number of pairs of inputs and outputs used to train the net, and also represents the size of the full pattern that the net is to learn; P represents the size of the input vector Xₘ or the number of input patterns of size M that the net learns to identify. Accordingly, each input vector Variable Xₘ has a size M. The number of samples M is frequently associated with a concept of time or epochs, because during the training of the net, the vectors will be shown over and over again.

With most problems it can be assumed that the input vectors Xᵢ (i=1, 2, . . . P) are not independent among themselves (i.e., Xᵢ*Xₖ is not zero). Establishing that, in a more general sense, the training input vectors are not orthogonal among themselves, and establishing that, for all Δ-rule variations used in backpropagation the weights are updated as a function of the input and output vectors, it can be assumed that the weights-variation-with-time connecting the inputs to a single neuron will not be orthogonal among themselves. Considering one neuron, k, in the first hidden-input layer, the input to the neuron at a given “time”, m, is

$${\sum }_{i = 0}^{P} ( {W}_{j , i , m} * {x}_{mi} )$$

and in general

$${\sum }_{i = 0}^{P} ( {W}_{j , i ,} * {X}_{j} ) .$$

W_{j,i} represents the vector “weight evolution” (of size M for a single training cycle) connecting input vector Xᵢ and neuron j. In the most general case vectors W_{j,i} are not orthogonal among themselves, i.e., Wⱼᵢ*Wⱼᵢ₊₁ is not equal to zero. This statement implies that the weights are not the independent variables, but vectors reflecting their “time-evolution”. Time is the evolution of the input signal, i.e., the changes in m, m=1,2 . . . M.

When a BP NN is used, independent of the method chosen to update the weights (steepest descendent, gradient, etc.), the derivative of the neuron inputs with respect to the weights is considered as

$$\frac{\partial ( {W}_{j} * {X}_{m} )}{\partial {W}_{j}} = {X}_{m}$$

where the vector Wⱼ indicates the weights inputting neuron j at a given time, m, and input vector Xₘ represents a collection of input vectors {xₘ₁, xₘ₂, xₘ₃ . . . x_{mN}} at a given time m, m=1,2 . . . M.

Rewriting the input neuron k over the whole time sequence, M, while the net is being trained, yields:

$${\sum }_{i = 1}^{P} \frac{\partial {W}_{j , i} * {X}_{i}}{\partial {W}_{j , i}} = {HX}_{i} \qquad ( 24 )$$

where H is a matrix containing the partial derivative of the weight vectors among themselves. The matrix H has 1 in the diagonal, and is an inverse-symmetric matrix, i.e., the top triangle is equal to 1/bottom-triangle.

H matrix represents the weights signature connecting the input vector Xₘ to the k neuron in the first hidden layer. If the weight-vectors Wⱼₘ were orthogonal, the matrix H will be identical to the identity matrix, and the resulting Fast-BP will be identical to the traditional BP.

From a mathematical point of view, to derivate with respect to a dependent variable is strictly incorrect; instead, the dependent variable should be written as function of the independent variables. For example, each weight vector connecting the input vector Xᵢ (of size P) to a given neuron j (in the hidden layer) should be written as a function Wⱼᵢ=f_{j,i}(t) where t represents the evolution of input vector Xᵢ, at different “time” m; m=1, 2 . . . M. The Chain Rule is then used and the correct derivative for each neuron j would be written as

$${\sum }_{i = 1}^{P} \frac{\partial {W}_{j , i} * {X}_{i}}{\partial {W}_{j , i}} = H * {X}_{i} = {\sum }_{i = 1}^{P} \frac{\partial {f}_{j , i} ( t ) * {X}_{i}}{\partial t} ( \frac{1}{\frac{\partial {f}_{j , i} ( t )}{\partial t}} ) \qquad ( 25 )$$

The function fj(t) is not known in advance; only fj(t) at time t and previous times are known. Therefore, a method such as the backward differentiation method or Euler method is used to calculate the derivative of the function with respect to the time. To accelerate the training the matrix H was used in the learning rule as indicated.

### Embodiments

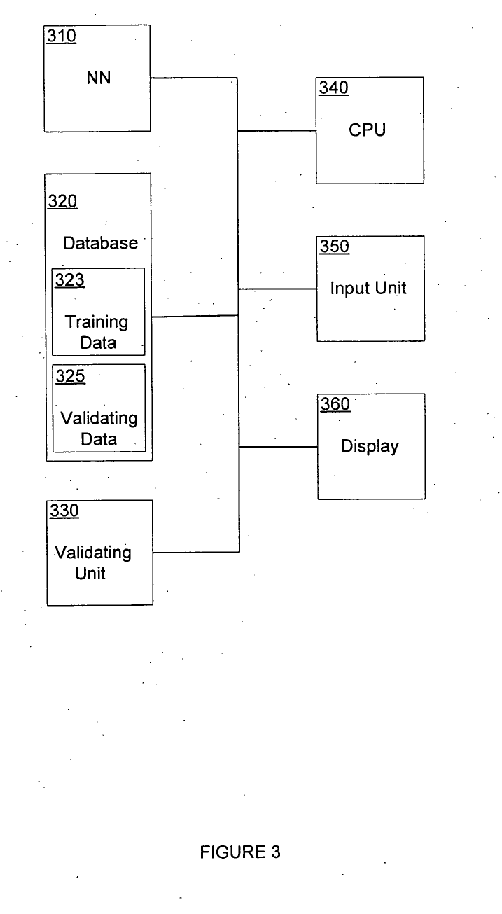

FIG. 3 illustrates one embodiment of the invention, wherein the neural network effect predictor (NNEP) 300 comprises a neural network (NN) 310, a database 320, a validating unit 330, a central processing unit (CPU) 340, and input unit 350, and a display 360. NN 310 is preferably an artificial neural network implemented in a computer programming language such as C++ or Matlab®, and is executed by CPU 340. Alternatively, the NN 310 is implemented in a hardware device such as a semiconductor chip. Database 320 comprises training data 323 for training the NN 310 and validating data 325 for validating the pharmacodynamic predictions of the NN 310 in the validating unit 330. Validating unit 330 is preferably implemented as a software component and compares the validating data 325 to the output of the NN 310 to determine the error in the NN 310. CPU 340 executes the NN 310 and the validating unit 330, and reads and writes to database 320. Input unit 350 allows training data and validation data to be input and written to the database 320. Display 360 displays the results of the NN 310 and the validating unit 330, as well as the contents of database 320.

The training data 323 includes drug dose data, drug effect data, and patient characteristics data for a plurality of patients from actual patient medical histories. The number of data sets necessary for the invention to operate with an acceptable error rate will vary, and may be easily determined through experimentation as is known in the art. The drug dose data and patient characteristics data are used as inputs for the NN 310, whereas the drug effect data is used by the NN 310 to calculate error and thus adjust the weights of the neural network. The drug dose data is represented a drug dose vs. time signature, which is vector of size 20 corresponding to 20 drug dose samples measured at time t=0, 0.016, 0.1, 0.15, 0.2, 0.5, 1, 12, 24, 36, 37, 48, 49, 72, 73.25, 120, 168, 216, 288, and 360 hours. Each entry in the vector is normalized to a value between 0 and 1. Accordingly, the time is neither an input nor an output, and drug dose data for each measured time is input to the NN 310 in parallel.

The patient characteristics data is represented as a vector of size 24, which contains the individuals clinical characteristics in the following order: Ethnicity as a 2 element binary description (i.e., 01 was used to assign white ethnicity, 10 to assign African American ethnicity, 11 assigned for Hispanic ethnicity, and 00 for Asian ethnicity), sex was assigned 1 for male and 0 for female, age was given in year (in addition to age the following “functional links” were added: age², age^{0.5}, age³, age^{0.33}, log₁₀ (age)), weight in Kg, stable angina (0 no, 1 yes), existence of previous myocardial infarction (MI) (0 no, 1 yes), history of diabetes (0 no, 1 yes), history of high blood pressure (o no, 1 yes), high cholesterol level (0 no, 1 yes), history of smoking (0 no, 1 yes, 0.5 yes in the past), prior percutaneous transhepatic cholangiogram (PTC) (0 no, 1 yes), prior carotid artery bruit (CAB) (Q no, 1 yes), use of Ticlid or Clopid (0 no, 1 yes), use of Statin (0 no, 1 yes), use of beta blockers (0 no, 1 yes), use of nitrates (0 no, 1 yes), use of a calcium channel blocker (CCB) (0 no, 1 yes), and use of a diuretic (0 no, 1 yes).

The drug effect data is represented in a drug effect vs. time signature, which is a vector of size 20 containing the sample drug effect at time t=0, 0.016, 0.1, 0.15, 0.2, 0.5, 1, 12, 24, 36, 37, 48, 49, 72, 73.25, 120, 168, 216, 288, and 360 hours. Thus time is neither an input or an output, and the drug effect data for each measured time is input to the NN 310 in parallel.

Validating data 325 is of the same format as training data 323. However, validating data is not used to train the NN 310.

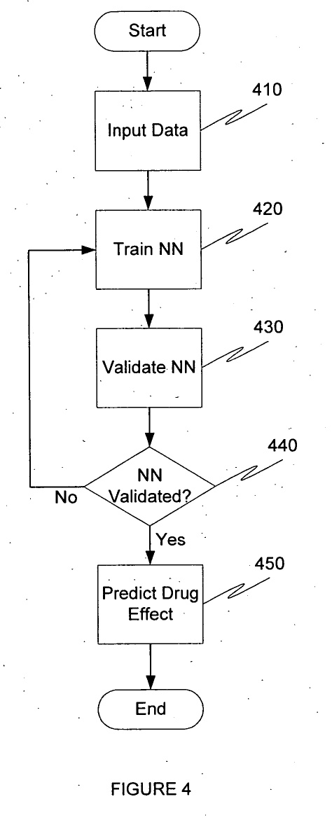

The operation of the NNEP is now described with reference to FIG. 4. Training data is input to the NN at step 410. At step 420, the NN is trained on the data sets. During the training process, the connections between neurons—or weights—(equivalent to the strength of the connection between the dendrites of biological neurons) are “adapted” by the mean of a “learning rule.” In the present embodiment, a steepest descent algorithm is used for the learning rule. However, the choice of one technique over the other is a balance between computer memory and computer training time, as can be determined by one of ordinary skill in the art. During the learning process, the NN “learns” solutions to a problem by changing its connection-weights in an iterative processing manner. The strength of the connection between two neurons is changed and adjusted each time that a training pair (input, output) is shown. In the present invention, both input and target-output data sets are given, and when the net output is calculated, it is compared to the given target-output. The resulting error, which is the difference between the two outputs (the net output and the target-output, or measured output), is then calculated and fed back to the network so that the weights can be adjusted and thus the error minimized. The weight changes throughout the whole network until the error for the entire training input set is at or less than a predefined level.

The transfer function used in each neuron (f(NET)) of the present embodiment is the hyperbolic tangent (TANH), which produces an output between −1 and 1. The data (inputs and outputs) are normalized between −1 and 1 (many input datum points have a value of 0, and if normalized between 0 and 1, those points will be assigned to 0, which itself does not carry information during the training process; by using bipolar normalization (between −1 and 1) the value of 0 is assigned −1, which will carry information). In constructing the NN, one, two, and three layers of nodes may be used for the NN. However, in the present embodiment a net using two layers provides the best performance with respect to the time required for lowering the normalized-average-error of the NN (output and target-output) to an acceptable level, such as +/−5%. Once an acceptable error rate is achieved, the NN weights are fixed.

After the NN has been trained on the data sets, the NN is validated at step 430. Validation is performed by inputting validating data to the trained NN. This validating data, like the training data, include drug dose data, drug effect data, and patient characteristics data for a plurality of patients from actual patient medical histories. However, the NN has not yet seen the validating data. The drug dose data and patient characteristics data are input into the NN as was done with the training data. The NN then outputs a predicted drug effect, however the NN does not compare predicted effect to the drug effect data to adjust the weights. Instead, the validating unit compares the drug effect predicted by the NN to the drug effect data to determine what, if any, error exists, thereby validating the efficacy of the NN.

At step 440, it is determined whether the validating unit validated the NN. If the validating unit validates the NN, i.e. if the NN predicted drug effect with an acceptable error, the process proceeds to step 450. If the validating unit did not validate the NN, more training is required and the process begins again at step 420.

Once an effective NN has been trained and validated, the NN may then be used to predict pharmacodynamic behavior for a specific patient at step 450. The specific patient's patient characteristics data is input to the NN along with an estimated dose. The NN outputs a predicted drug effect based on the specific patient's medical history and the estimated dose, thereby allowing a doctor to determine whether the desired drug effect may be achieve with the estimated dose. This step is then iterated with adjustments to the estimated dose until the desired drug effect is achieved.

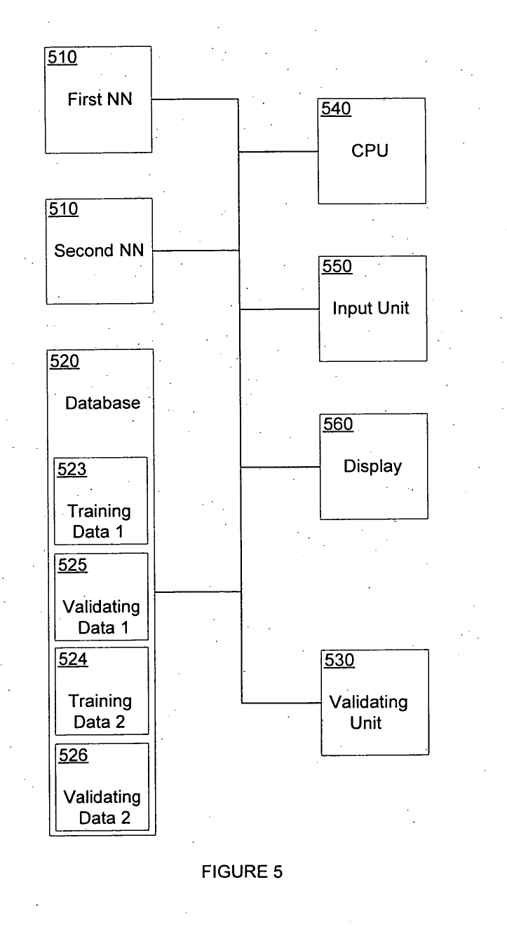

FIG. 5 illustrates another embodiment of the invention, wherein the neural network dosage predictor (NNDP) 500 comprises a first NN 510, a second NN 515, a database 520, a validating unit 530, a central processing unit (CPU) 540, and input unit 550, and a display 560. NN 510 and NN 515 are preferably artificial neural networks implemented in a computer programming language such as C++ or Matlab®, and are executed by CPU 540. Alternatively, the NN 510 and NN 515 are implemented in a hardware device such as a semiconductor chip. Database 520 comprises first training data 523 for training the first NN 510, second training data 524 for training the second NN 515, first validating data 525 for validating the pharmacodynamic predictions of the first NN 510 in the validating unit 530, and second validating data 526 for validating the dosage predictions of the second NN 515. Validating unit 530 is preferably implemented as a software component and compares the first validating data 525 to the output of the first NN 510 and the second validating data 526 to the output of the second NN 515 to determine the error in the NN 510 and the NN 515. CPU 540 executes the NN 510, the NN 515, and the validating unit 530, and reads and writes to database 520. Input unit 550 allows training data and validation data to be input and written to the database 520. Display 560 displays the results of the NN 510, the NN 515, and the validating unit 530, as well as the contents of database 520.

The first training data 523 and the second training data 524 both include drug dose data, drug effect data, and patient characteristics data for a plurality of patients from actual patient medical histories. The number of data sets necessary for the invention to operate with an acceptable error rate will vary, and may be easily determined through experimentation. The drug dose data and patient characteristics data are used as inputs for the first NN 510, whereas the drug effect data is used by the first NN 510 to calculate error and thus adjust the weights of the first NN. The drug effect data and patient characteristics data are used as inputs for the second NN 515, whereas the drug does data is used by the second NN 515 to calculate error and thus adjust the weights of the second NN. The drug dose data is represented as a drug dose vs. time signature, which is a vector of size 20 corresponding to 20 drug dose samples measured at time t=0, 0.016, 0.1, 0.15, 0.2, 0.5, 1, 12, 24, 36, 37, 48, 49, 72, 73.25, 120, 168, 216, 288, and 360 hours. Each entry in the vector is normalized to a value between 0 and 1. Accordingly, the time is neither an input nor an output, and drug dose data for each measured time is input to the NN in parallel.

The patient characteristics data is represented a vector of size 24, which contains the individuals clinical characteristics in the following order: Ethnicity as a 2 element binary description (i.e., 01 was used to assign white ethnicity, 10 to assign African American ethnicity, 11 assigned for Hispanic ethnicity, and 00 for Asian ethnicity), sex was assigned 1 for male and 0 for female, age was given in year (in addition to age the following “functional links” were added: age², age^{0.5}, age³, age^{0.33}, log₁₀ (age)), weight in Kg, stable angina (0 no, 1 yes), existence of previous MI (0 no, 1 yes), presence of diabetes (0 no, 1 yes), high blood pressure (0 no, 1 yes), high cholesterol level (0 no, 1 yes), history of smoking (0 no, 1 yes, 0.5 yes in the past), prior PTC (0 no, 1 yes), CAB (0 no, 1 yes), use of Ticlid or Clopid (0 no, 1 yes), use of Statin (0 no, 1 yes), use of beta blockers (0 no, 1 yes), use of nitrates (0 no, 1 yes), use of a CCB (0 no, 1 yes), and use of a diuretic (0 no, 1 yes).

The drug effect data is represented in a drug effect vs. time signature, which is a vector of size 20 containing the sample drug effect at time t=0, 0.016, 0.1, 0.15, 0.2, 0.5, 1, 12, 24, 36, 37, 48, 49, 72, 73.25, 120, 168, 216, 288, and 360 hours. Thus time is neither an input or an output, and the drug effect data for each measured time is input to the NN in parallel.

The first validating data 325 and the second validating data 326 are of the same format as first training data 323 and second training data 324. However, validating data is not used to train the NNs.

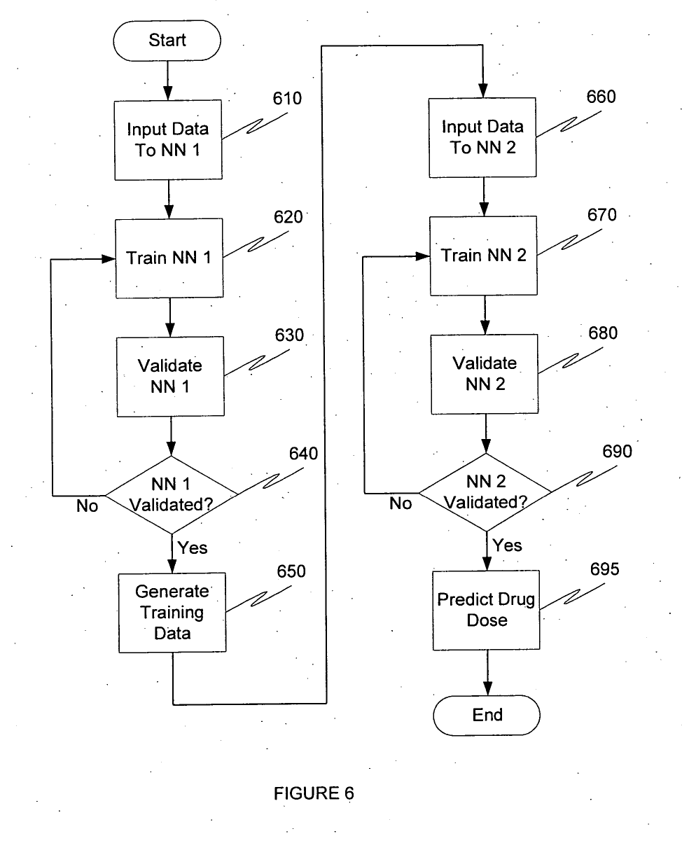

The operation of the NNDP 500 is now described with reference to FIG. 6. First training data is input to the first NN at step 610. At step 620, the first NN is trained on the data sets. The training process is the same as was described with reference to FIG. 4. After the first NN has been trained on the data sets, the first NN is validated at step 630. Validation is performed by inputting the first validating data to the trained NN. This first validating data, like the training data, includes drug dose data, drug effect data, and patient characteristics data for a plurality of patients from actual patient medical histories. However, the first NN has not yet seen the validating data. The drug dose data and patient characteristics data are input into the first NN as was done with the first training data. The first NN then outputs a predicted drug effect, however the first NN does not compare predicted effect to the drug effect data to adjust the weights. Instead, the validating unit compares the drug effect predicted by the first NN to the drug effect data to determine what, if any, error exists, thereby validating the efficacy of the first NN.

At step 640, it is determined whether the validating unit validated the first NN. If the validating unit validates the first NN, i.e. if the first NN predicted drug effect with an acceptable error, the process proceeds to step 650. If the validating unit did not validate the first NN, more training is required and the process begins again at step 610.

Once the first NN has been trained and validated, the first NN is then used to generate the second training data for the second NN at step 650. The second NN is an inverse of the first NN. That is, instead of mapping patient characteristics and drug dose to pharmacodynamic behavior as in the first NN, the second NN maps patient characteristics and pharmacodynamic behavior to drug dose. Rather, instead of predicting drug effect for a drug dose, the second NN predicts a drug dose given a desired drug effect. The second training data is generated by inputting hypothetical patient characteristics and a drug dose to the first NN, which generates a predicted drug effect. Accordingly, the second NN can be trained with large number of samples without the need for a large number of clinical studies. Preferably, the second training data also comprises data from actual patients.

The second training data is input to the second NN at step 660. At step 670, the second NN is trained on the second training data. The training is the same as described with reference to the previous embodiment.

The transfer function used in each neuron (f(NET)) of the present embodiment is the hyperbolic tangent (TANH), which produces an output between −1 and 1. The data (inputs and outputs) are normalized between −1 and 1 (many input datum points have a value of 0, and if normalized between 0 and 1, those points will be assigned to 0, which itself does not carry information during the training process; by using bipolar normalization (between −1 and 1) the value of 0 is assigned −1, which will carry information). In constructing the second NN, one, two, and three layers of nodes may be used for the second NN. However, in the present embodiment a net using three layers provides the best performance with respect to the time required for lowering the normalized-average-error of the second NN (output and target-output) to an acceptable level, such as +/−5%. Once an acceptable error rate is achieved, the second NN weights are fixed.

After the second NN has been trained on the data sets, the second NN is validated at step 680. Validation is performed by inputting second validating data to the second NN. This validating data, like the training data, includes drug dose data, drug effect data, and patient characteristics data for a plurality of patients from actual patient medical histories. However, the second NN has not yet seen the second validating data. The drug effect data and patient characteristics data are input into the second NN as was done with the training data. The second NN then outputs a predicted drug dose, however the second NN does not compare predicted dose to the drug dose data to adjust the weights. Instead, the validating unit compares the drug dose predicted by the second NN to the drug dose data to determine what, if any, error exists, thereby validating the efficacy of the second NN.

At step 690, it is determined whether the validating unit validated the second NN. If the validating unit validates the second NN, i.e. if the second NN predicted drug dose with an acceptable error, the process proceeds to step 695. If the validating unit did not validate the second NN, more training is required and the process begins again at step 670.

Once an effective second NN has been trained and validated, the second NN is then used to determine a drug dose for a specific patient at step 695. The specific patient's patient characteristics data is input to the second NN along with a desired effect. The second. NN outputs a predicted drug dose based on the specific patient's medical history and the desired effect.

### EXAMPLES

### Example 1

### Predicting Pharmacodynamic Behavior of Abciximab

Abciximab is an antagonist of the platelet GPIIb/IIIa receptor and is effective in preventing coronary thrombosis following percutaneous transluminal coronary angioplasty (PTCA). Clinical dose of abciximab is based on achieving >80% GP IIb/IIIa receptor blockade and inhibition of ex vivo platelet aggregation induced by 20 μM ADP to 20% of baseline values. This is achieved by administration of an initial weight-corrected bolus dose followed by an intravenous infusion in some studies. Maximum inhibition of platelet function and receptor occupancy of the external pool of GPIIb/IIIa occurs quickly (within three minutes) following abciximab administration, and abciximab effect continues for the life of the platelet, with offset of effect being partly the result of platelet turnover. Following discontinuation of the drug, there is a gradual decline in receptor occupancy over 15 days consistent with the appearance of new platelets.

Abciximab dose-plasma concentration-effect relationships were determined from three separate clinical studies: one study of 30 healthy subjects ages 21-66 (set No. 1); and two independent studies (set No. 2 with 32 patients, and set No. 3 with 15 patients) on patients undergoing PTCA.

Set No. 1. Healthy Individuals.

This study was conducted at the Georgetown University Medical Center Clinical Research Center. Thirty healthy volunteers ages 21-66 participated. Each subject ingested aspirin (325 mg) by mouth at least 4 but not more than 24 hours prior to initial abciximab exposure. At study time 0 a 0.25 mg/kg intravenous bolus of abciximab was administered, immediately followed by a 0.125 μg/kg/min intravenous abciximab infusion for the following 24 hours, at which time the abciximab infusion was stopped. To this point the protocol was identical for each of the study groups. The first treatment group (Group 1) then received 0.05 mg/kg intravenous abciximab bolus doses every 15 minutes to a cumulative dose of 0.25 mg/kg starting 24 hours after cessation of the abciximab infusion (48 hours following the initial abciximab bolus dose). The second treatment group (Group 2) received 0.025 mg/kg intravenous abciximab bolus doses every 15 minutes to a cumulative dose of 0.1 mg/kg starting 12 hours after cessation of abciximab infusion (36 hours following the initial abciximab bolus dose). The third treatment group (Group 3) received 0.05 mg/kg intravenous abciximab bolus doses every 15 minutes to a cumulative dose of 0.25 mg/kg starting 48 hours after cessation of abciximab infusion (72 hours following the initial abciximab bolus dose).

Blood samples for determination of abciximab concentration and pharmacodynamic measurement (platelet aggregation), drawn into tubes containing citrate anticoagulant, were obtained at baseline (within 2 hours prior to administering the first abciximab bolus dose), at 6, 12, 18, and 24 hours following the initial bolus, and at either 4-hour intervals (Groups 1 and 2) or 8-hour intervals (Group 3) until administration of the second series of abciximab bolus infusions. Samples were then obtained immediately prior to each bolus and at 15 minutes following administration of the last bolus.

Set No. 2. Patients Undergoing Elective PTCA.

This study was conducted involving patients undergoing PTCA at the Baylor College of Medicine affiliated hospitals, The Methodist Hospital, and Ben Taub Hospital. Thirty-two patients ages 44-74 participated. Patients who were scheduled to undergo elective PTCA were enrolled after providing written informed consent for the protocol, which was approved by the Baylor College of Medicine, The Methodist Hospital, and the Ben Taub Hospital IRB's. Each patient ingested (orally) aspirin (325 mg) at least 2 hours but not more than 6 hours prior to abciximab administration. After vascular access was established in the catheterization laboratory, each patient was administered a 12,000-unit bolus of unfractionated heparin intravenously, followed by repeat boluses of heparin to maintain an activated clotting time of 300-400 seconds during the procedure. At least 15 minutes following initiation of heparin therapy and 2-60 minutes prior to angioplasty balloon inflation, a single 0.25 mg/kg intravenous bolus dose of abciximab was administered. Heparin administration was continued for at least 6 hours following the procedure. Blood samples for determination of abciximab concentrations, drawn into tubes containing citrate anticoagulant, were obtained as follows: the first sample 15-120 minutes prior to abciximab, then samples immediately prior to abciximab, and at 2, 5, 10, 20, 30 minutes, and 1, 2, 4, 6, 8, 12, 24, and 48 hours following abciximab administration. Blood samples for determination of ADP stimulated platelet aggregation and determination of GP IIb/IIIa receptor occupancy were obtained prior to heparin administration, immediately prior to abciximab administration (post heparin administration), and at 2, 6, and 24 hours post abciximab administration. In 12 randomly selected patients additional samples at 4, 8, and 48 hours post abciximab administration were obtained.

Set No. 3. Patients Undergoing PTCA.

This study was conducted involving 15 patients undergoing PTCA at St. James's Hospital, Dublin, Ireland. Patients between the ages of 21 and 70 with clinically significant coronary artery disease suitable for coronary angioplasty participated in the study after obtaining written informed consent. The protocol was reviewed and approved by the Irish Medicine Board and the Ethics Committee of St. James's Hospital.

Patients received a bolus (0.25 mg/kg) followed by a 36-hour infusion (0.125 mg/kg/min to a maximum of 10 mg/min) of abciximab 18 to 24 hours before elective coronary intervention. Unfractionated heparin was administered as a bolus (50-70 U/kg to a maximum of 7000 U). All patients received 300 mg of aspirin 4 hours before the procedure. Patients who had a coronary stent inserted received an ADP receptor antagonist (250 mg of ticlopidine b.i.d. or 75 mg of clopidogrel daily) starting immediately following the procedure and this was continued for 4 weeks following procedure.

Blood samples were collected from a peripheral vein into 3.8% sodium citrate at a final dilution of 1 in 10. Samples were collected at baseline (day 1); before the abciximab bolus; and at 1, 3, 5, 10, 30, and 60 minutes, and 12, 24, and 36 hours after the initial bolus of abciximab. Additional samples were drawn on days 3, 5, 7, 9, 12, and 15.

GP IIb/IIIa Receptor Occupancy Assay

The total number of baseline abciximab receptors and the degree of GP IIb/IIIa receptor blockade at post-initial abciximab treatment times were quantified by the radiometric method. The percent GP IIb/IIIa receptor blockade was calculated as follows:

$$\frac{\begin{matrix}( Baseline GPIIb / IIIa receptor number - \\ Post Treatment Unoccupied Receptors ) \times 100\end{matrix}}{( Baseline GPIIb / IIIa receptor number )} \qquad ( 26 )$$

Platelet Aggregation

Inhibition of platelet aggregation was evaluated by the turbidimetric method. The extent of platelet aggregation was quantified as the maximum change in light transmittance at 4 minutes after addition of the ADP antagonist. For each sampling time, the percent baseline aggregation was determined by the following calculation:

$$\frac{\begin{matrix}( Maximum Change in Light \\ Transmittance of Test Sample ) \times 100\end{matrix}}{\begin{matrix}( Maximum Change in Light \\ Transmittance of Baseline Sample )\end{matrix}} \qquad ( 27 )$$

Results

Those skilled the art of neural networks will appreciate that there is no absolute formula for determining the number of neurons to use for a particular application. The number of layers and neurons depends greatly on the number of inputs used, the complexity of the mapping, and the hardware implementing the neural network. Consequently some experimentation will be necessary to determine an optimal system. However, using a 1.3 GHz PC, the inventors preferred an implementation using a 2-layer BP NN with 100 neurons in the first layer and 100 in the second layer. The 2-layer BP NN was trained using the abciximab dose-time signature and subject or patient medical history as inputs, and the percent inhibition of 20 μM ADP-induced platelet aggregation versus time as the output. The database used for training the net contained all healthy individuals (Set No. 1) and 8 patients from Set No. 3. Seven patients from Set No. 3, and all patients from Set No. 2 were excluded from NN training to be used subsequently for validation of the trained system. The healthy subjects were included in the training set in order to “teach” the NN the difference between healthy subject medical history, and the medical history of the patients undergoing angioplasty. The adopted data representation for the time signatures was that of 20 points time signature of dose (as input), and 20 points time signature of percentile baseline 20 μM ADP-induced platelet aggregation. Dose and percent baseline platelet aggregation ADP signatures were measured at the following sampling times: 0, 0.016, 0.05, 0.083, 0.1666, 0.5, 1, 12, 24, 36, 37, 48, 72, 73.25, 120, 168, 216, 288, and 360 hours. During the learning process the epochs were set at one (epoch=1), meaning that every time an input vector is shown to the net, the error was calculated and the weights immediately updated. After training the net for 48 hours on a 1.3 GHz PC, the minimum error reached by the net—on a 0-1 scale—was of 0.04 (4%) on average (range 2-9%).

After the net was trained the weights remained fixed. By exploring the inputs that had a greater contribution to the learning of the NN (higher weight values)—in addition to the expected impact of the dose-time signature—the inventors found that age, ethnicity, nitrates, β-blockers, statins, smoking, and high blood pressure were the input variables that greatly impacted learning, with age being most important.

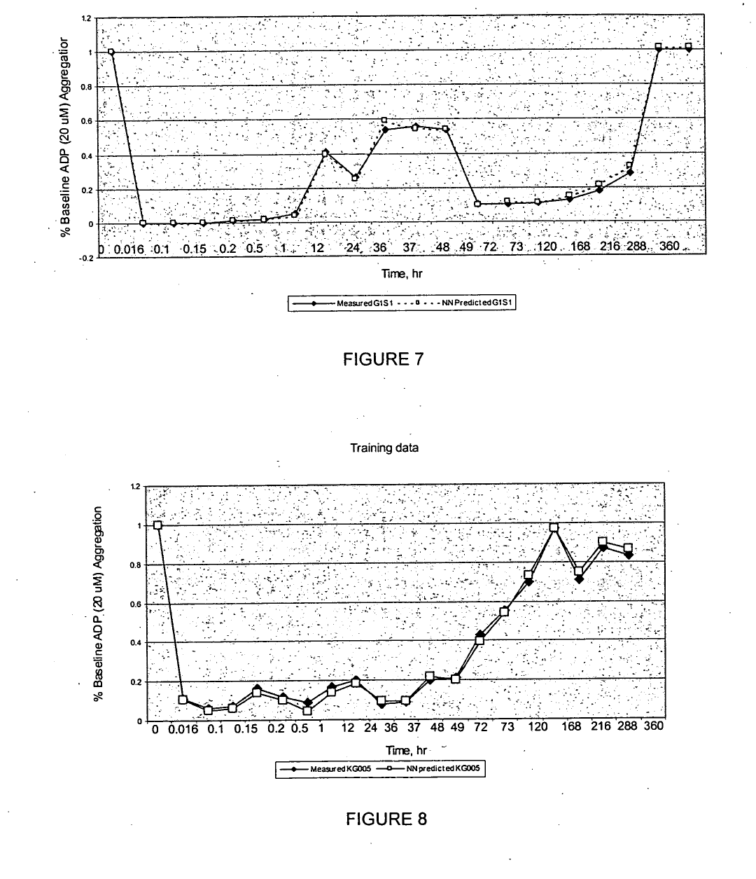

FIGS. 7 and 8 show a comparison between the % baseline ADP (20 μM) aggregation versus time that the NN calculated and the measured data. Healthy individuals' drug responses are shown in FIGS. 7 and a patient response is shown in FIG. 8. It can be seen that the two lines (in each figure) are virtually identical.

The NN capabilities were validated by inputting only the dose-signature at the times indicated above and the patient history as indicated in Table 1.

TABLE 1
Individual and Patient Characteristics

| Subject and Patient | Set No. 1 | Set No. 2 | Set No. 3 |
|---|---|---|---|
| Characteristics | N = 30 | N = 32 | NA = 15 |
| Ethnicity (B/W/H/A)* | 16/13/0/1 | 5/22/4/1 | 0/15/0/0 |
| Sex (M/F) | 28/2 | 20/12 | 12/3 |
| Age (mean ± SD) (years) | 40 ± 10 | 58 ± 9 | 57 ± 7 |
| Weight (mean ± SD) (Kg) | 84 ± 18 | 84 ± 18 | 72 ± 13 |
| Stable angina (y/n) | 0/30 | 12/20 | 4/11 |
| Previous MI (y/n) | 0/30 | 8/24 | 5/10 |
| Diabetes (y/n) | 0/30 | 4/28 | 1/14 |
| Hypertension (y/n) | 0/30 | 7/25 | 4/11 |
| Hypercholesterolemia (y/n) | 0/30 | 2/30 | 3/12 |
| Smoking (y/n) | 0/30 | 9/23 | 7/8 |
| Prior PTCA (y/n) | 0/30 | 9/13 | 3/12 |
| Prior CABG (y/n) | 0/30 | 11/21 | 1/14 |
| Ticlid or Clopid (y/n) | 0/30 | 7/25 | 12/3 |
| Statins (y/n) | 0/30 | 9/23 | 6/9 |
| β-blocker (y/n) | 0/30 | 31/1 | 11/4 |
| Nitrates (y/n) | 0/30 | 31/1 | 2/13 |
| Calcium antagonists (y/n) | 0/30 | 4/28 | 1/14 |
| Diuretics (y/n) | 0/30 | 6/26 | 1/14 |

*B - African American; W - Caucasian; H - Hispanic; A - Asian

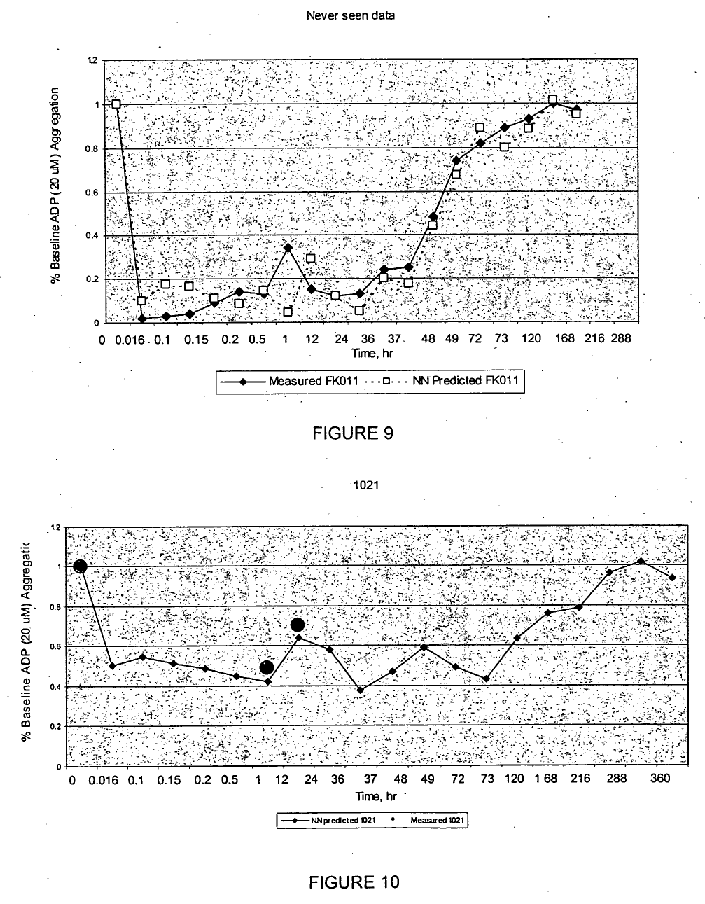

FIGS. 9 and 10 show the predicted response calculated by the trained net (in FIG. 9 the solid line represents the measured data and the broken line represents the NN predictions on patients from data Set No. 3; and in FIG. 10 the dots represent the measured data and the broken line represents the NN predictions on patients from data Set No. 2). A small number of platelet aggregation measurements were available for each patient in data Set No. 2. The predictive performance of the NN was measured by calculating the correlation coefficient for all the data of patients “never seen” by the net. This comparison was performed only for data Set No. 3 for which detailed measured information was available. As shown in FIGS. 9 and 10, the NN predictions coincides with the measured datum points. The correlation coefficient (in a scale 0-1, 1 indicating perfect correlation) between the two vectors—measured data, and NN-predicted data—which provided a measure of how close the two vectors (lines) were, was calculated for each individual and then averaged over all samples (individuals) tested, resulting in a mean of 0.86 an a standard deviation of 0.08. Correlation coefficient of the area under the curve; i.e., % baseline 20 μM ADP-induced platelet aggregation versus time give a mean correlation coefficient of 0.98 and a standard deviation of 0.02. Comparing the correlation coefficients of the two curves (0.86 and 0.98) indicates that the major difference is at times away from time zero, when the bolus was administered.

The correlation coefficient between two vectors, X and Y, is calculated as follows:

$${r}_{x , y} = \frac{Cov ( X , Y )}{{σ}_{x} {σ}_{y}} \qquad ( 28 )$$

where −1<r_{xy}<1, and the covariance is defined as

$$Cov ( X , Y ) = \frac{1}{n} {\sum }_{1}^{n} ( {x}_{i} - {μ}_{x} ) ( {y}_{j} - {μ}_{y} ) \qquad ( 29 )$$

Where σₓ and σ_{y} represent the standard deviation of the vector X and Y, and μₓ and μ_{y} represent the mean value of the vector X and Y. Here X is the NN-predicted vector (set of values) and Y is the measured % baseline ADP (20 μM) aggregation.

Studies based on plasma-concentration/effect using a sigmoid Eₘₐₓ model calculated from PK/PD models for data Set No. 2 were calculated for the abciximab concentrations required to achieve ≧80% platelet glycoprotein (GP) IIb/IIIa receptor occupancy and ≧80% inhibition of ADP-induced platelet aggregation in patients undergoing PTCA at 100-175 ng/ml, based on a mean (±SD) calculated value of 141+16.8 ng/ml.

However prior to comparison of this calculation to the NN predictions, in order to validate the performance of the NN by independent means, it was necessary to convert the plasma concentration values shown above to drug effect. Accordingly, before comparing the NN results to the calculated plasma concentration (using traditional PK/PD), the plasma-concentrations were converted to percent inhibition of 20 μM ADP-induced platelet aggregation. To do so an apparent volume of distribution for abciximab must be estimated for each individual, defined as follows:
V=Amount-of-drug-in-the-body/concentration-measured-in-plasma (30)
The equations that apply are:
Cₚ=DOSE/V*EXP(−Kel*t) (31).
where Cp is the plasma concentration in mg/L; DOSE is the dose in mg; V is the apparent volume in liters; and t the time in hours. Cₚ⁰ is the plasma concentration extrapolated back to time 0 before drug administration.
Cₚ⁰=DOSE/V (32)
Kel is the elimination rate constant determined for the individual. If the dose administered is known, and the plasma concentrations at two (or more) times after a bolus is administered, and after distribution equilibrium has occurred, then V can be calculated. For this purpose equation (33) is derived:
ln Cp=ln Cp⁰−kel*t (33)
The apparent volume of distribution for abciximab can then be calculated using equations (31) and (33).

Patients in data set 2 were administered a single intravenous abciximab bolus at t=0, and plasma concentrations were measured over the next several hours. The calculated abciximab volume of distribution for the 32 patients in data set 2 was (mean±SD) 134±60.2 liters. Using the calculated apparent volume of distribution for abciximab, the estimated plasma concentration for these patients was used to calculate the corresponding mean required dose. The calculated mean dose was of 18.9±2.0 mg.

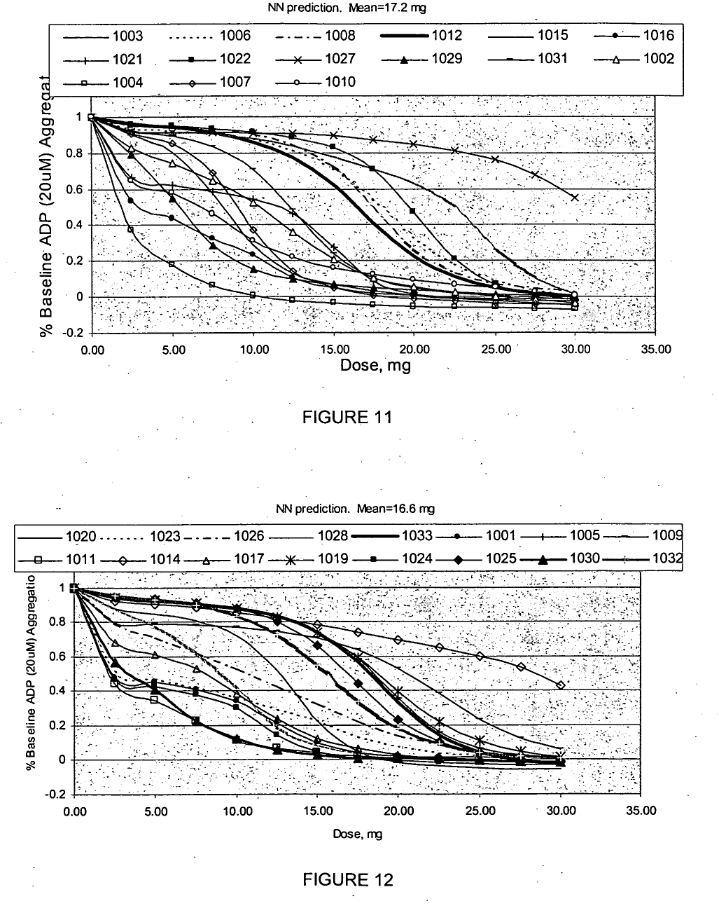

The inventors compared the corresponding dose required to maintain 80% inhibition of 20 μM ADP-induced platelet aggregation using a conventional pharmacodynamic model to the mean dose required to maintain the same level of platelet inhibition predicted using the NN pattern recognition. Results are summarized in FIGS. 11 and 12.

The trained NN accurately predicted the percent inhibition of 20 μM ADP-induced platelet aggregation signature over 15 days from the dose-time profile and the subjects' medical history, without the input of the plasma abciximab concentration. The NN model does not impose any physical or chemical hypothesis. Furthermore, the NN explored the impact—on the percent inhibition of platelet aggregation signature—of the previously determined and most important variables in the patients' medical history on prediction of the response. Aggregation-time profiles were calculated when different dose-time single bolus profiles were input.

### Example 2

### Predicting Abciximab Dose

The NN designed in the previous example was used to generate hypothetical data to train an inverse NN. The inverse NN performed the inverse job; i.e., given the patient history and desired effect that the physician would like the drug to have on the patient—in this example the % Baseline ADP (20 uM) Aggregation of platelets-vs.-time profile—the inverse NN was used to predict the dose profile needed to obtain the desired effect.

Several net topologies of a supervised backpropagation were tested. The most successful training was performed with a 3 hidden layer BP NN with 80 neurons per layer and using a TANH transfer function and data (input and output) normalized to ±1. The learning rule used was an extended delta bar with forgetting factor and momentum. During training, the weights between neurons were updated every time 5 samples were shown (epochs=5). During the training, a total of 200 input/output vector sample sets were used, including Set No. 1 with 20 samples (out of 30), Set No. 2 with 32, and Set No. 3 with 15 samples, giving a total of 67 samples. The remaining 133 samples were “artificially generated” by means of the NN designed to map the clinical history of the patient and the % Baseline ADP (20 uM) Aggregation of platelets vs. time profile into the dose versus time. The error (RMS) reached after 48 hours of training in a PC 900 MHz reached about +5%.

Once the net reached an acceptable error—within the experimental error, assumed to be ±5%—the training was stopped and the net was used to make hypothetical predictions oh individuals among the 3 sets that were not used during training. Tables 2 and 3 show the characteristics of the individuals used to test the net.

TABLE 2

| Patients | DQ0015 | EM0014 | EH0013 | SK002 | PC008 | PD001 |
|---|---|---|---|---|---|---|
| Ethnicity | 01 | 01 | 01 | 01 | 01 | 01 |
| Sex | 1 | 1 | 1 | 1 | 1 | 1 |
| Age (years) | 54 | 49 | 48 | 56 | 60 | 61 |
| Weight (Kg) | 82 | 70 | 83 | 70 | 95 | 70 |
| Stable Angina | 0 | 0 | 0 | 1 | 0 | 0 |
| (y = 1/n = 0) | | | | | | |
| Previous MI (y = 1/n = 0) | 0 | 1 | 1 | 0 | 0 | 0 |
| Diabetes (y = 1/n = 0) | 0 | 0 | 0 | 1 | 0 | 0 |
| HT (y = 1/n = 0) | 0 | 1 | 0 | 0 | 0 | 1 |
| Cholesterol (y = 1/n = 0) | 0 | 0 | 0 | 1 | 0 | 0 |
| Smoking | 0.5 | 1 | 1 | 1 | 1 | 0.5 |
| (y = 1/n = 0/before = 0.5) | | | | | | |
| Prior PTCA | 0 | 0 | 1 | 0 | 0 | 0 |
| (y = 1/n = 0) | | | | | | |
| Prior CAB | 0 | 0 | 0 | 0 | 0 | 0 |
| (y = 1/n = 0) | | | | | | |
| TICLID or CLOPID | 1 | 0 | 0 | 0 | 0 | 1 |
| (y = 1/n = 0) | | | | | | |
| Statins | 1 | 0 | 1 | 0 | 1 | 0 |
| (y = 1/n = 0) | | | | | | |
| b-Blocker | 1 | 1 | 1 | 0 | 1 | 1 |
| (y = 1/n = 0) | | | | | | |
| Nitrates | 1 | 1 | 0 | 0 | 0 | 0 |
| (y = 1/n = 0) | | | | | | |
| CCB | 0 | 0 | 0 | 0 | 0 | 0 |
| (y = 1/n = 0) | | | | | | |
| Diuretics | 0 | 0 | 0 | 0 | 0 | 0 |
| (y = 1/n = 0) | | | | | | |

Ethnicity: African American 10; White 01; Hispanic 11; Asian 00;
Sex: Female 0; Male 1

TABLE 3

| Patient | 1006 | 1022 | 1033 | 1019 | 1009 | G1S5 | G2S2 | G2S2 | G2S2 |
|---|---|---|---|---|---|---|---|---|---|
| Ethnicity | 11 | 01 | 01 | 11 | 01 | 01 | 01 | 01 | 01 |
| Sex | 1 | 1 | 1 | 1 | 1 | 1 | 1 | 1 | 1 |
| Age (years) | 52 | 60 | 60 | 51 | 44 | 33 | 66 | 66 | 66 |
| Weight (Kg) | 77 | 101 | 85 | 79 | 94 | 88.6 | 94.5 | 94.5 | 94.5 |
| General | Beta | Beta | Beta | Beta | Beta | Healthy: | Healthy: | Healthy: | Healthy: |
| Information | Blocker; | Blocker; | Blocker; | Blocker; | Blocker; | Not drugs | Not drugs | Not drugs | Not drugs |
| | Calcium | Calcium | Calcium | Calcium | Calcium | | | | |
| | Chan. | Chan. | Chan. | Chan. | Chan. | | | | |
| | Blocker; | Blocker; | Blocker; | Blocker; | Blocker; | | | | |
| | NTG-IV; | NTG-IV; | NTG-IV; | NTG-IV; | NTG-IV; IV | | | | |
| | Nitrates | Nitrates; | Nitrates | Nitrates | tPA | | | | |
| | | Diuretic | | | | | | | |

Ethnicity: African American 10; White 01; Hispanic 11; Asian 00
Sex: Female 0; Male 1

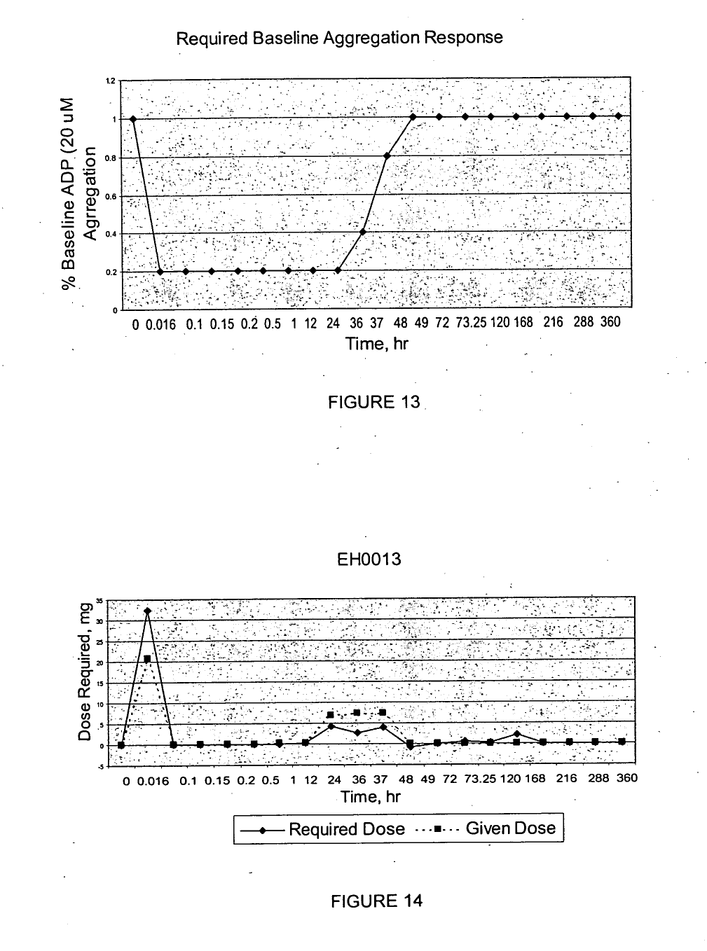

Two hypothetical required responses were defined: (1) as the dose needed to maintain a % baseline ADP (20 μM) aggregation of platelet to remain at 20% for 24 hrs (See FIG. 13); (2) as the dose needed to maintain a % baseline ADP (20 μM) aggregation of platelet to remain at 20% for 37 hrs. (See FIG. 20).

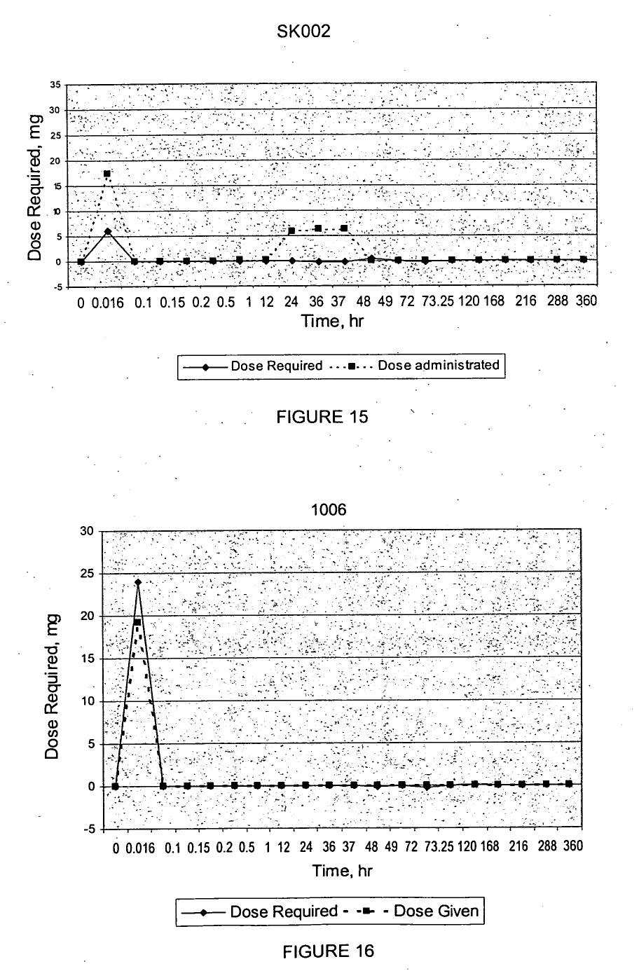

Then, the inverse-NN response of the required dose was compared to the dose that was administrated to those same patients. FIGS. 14 and 15 show the inverse-NN dose required (to maintain the dose profile as shown in FIG. 13) compared to the administrated dose for patients from Data Set No. 3 (see patients EH0013 and SK002 from Table 4); these patients were undergoing an angioplasty procedure. The solid line shows the NN recommended dose, while the dotted line shows the dose signature that was administrated to that individual. From the two individuals chosen, one had received a larger dose than the one indicated by the Inverse-NN (See FIG. 15) and other received a dose that would not keep his % baseline platelets at the 20% levels required for 24 hrs (See FIG. 14).

Similar results for a patient from Data Set No. 2 (see patient 1006 from Table 5) are shown in FIG. 16. Notice that the difference on dose for that patient is not as pronounced as the examples shown in FIGS. 14 and 15. This is possibly due to the fact that patients in Data Set No. 2 were sick individuals who were not yet scheduled to undergo angioplasty but were involved in a clinical trial, while individuals in Data Set No. 3 had been scheduled to have an angioplasty.

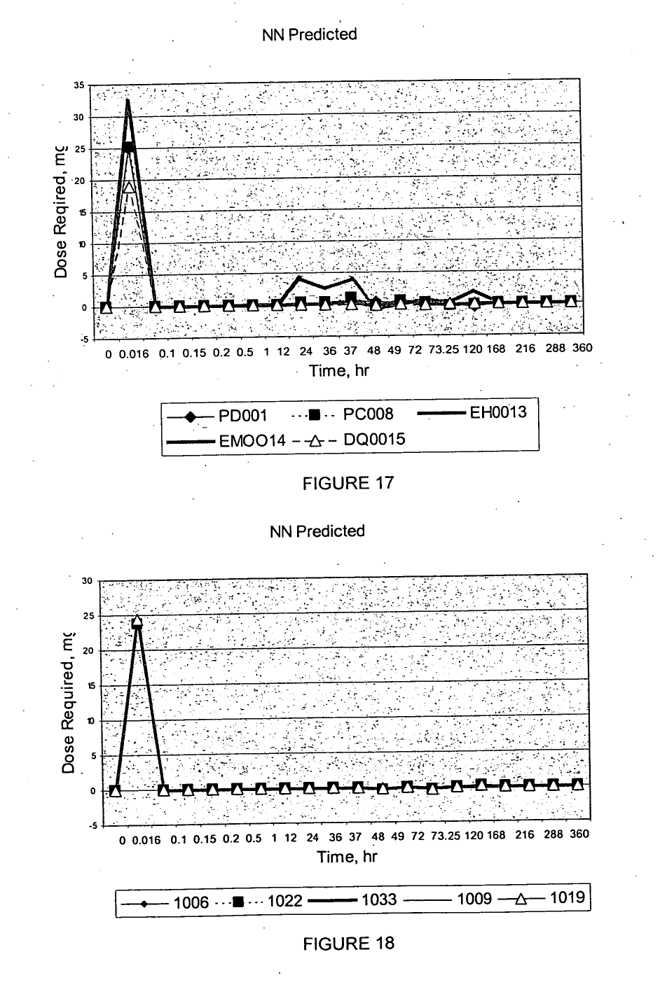

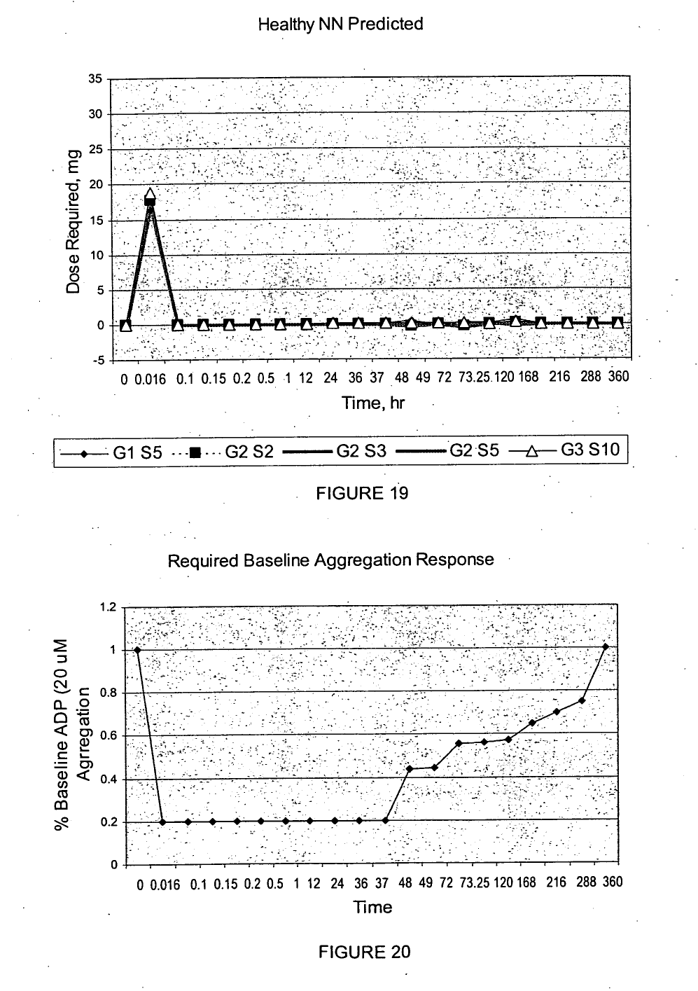

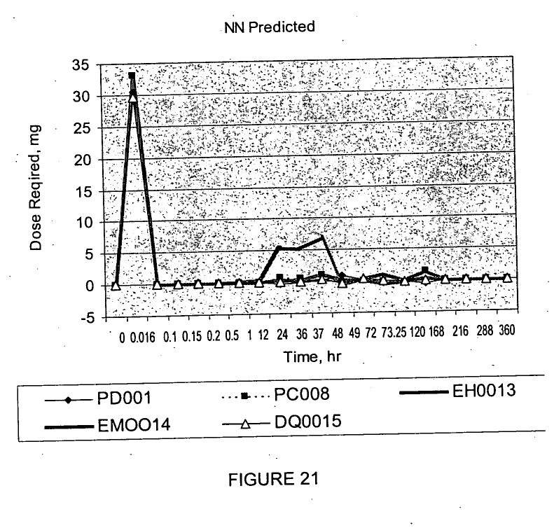

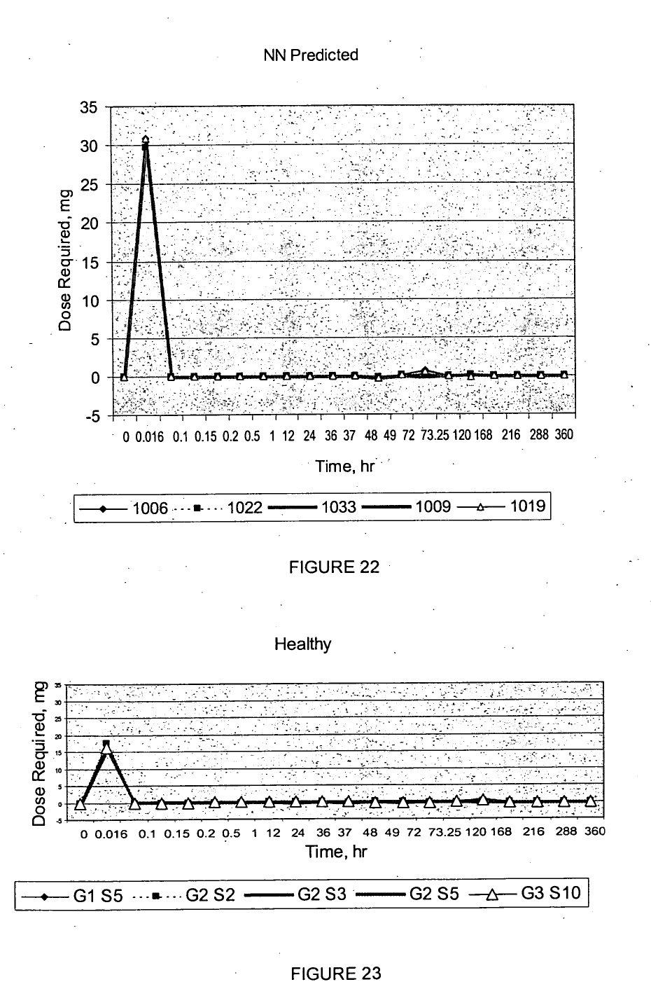

FIGS. 17 to 19 show the dose required to maintain a hypothetical % baseline ADP (20 μM) aggregation of platelet to remain at 20% for 24 hrs for individuals in Data Set No. 3, Data Set No. 2, and Data Set No. 1, respectively. All these dose calculations were performed with the trained NN.

FIG. 20 shows another hypothetical, but more “demanding,” drug effect time signature. Here it is required to maintain a % of baseline ADP (20 μM) aggregation equal to 20% for as long as 37 hrs. FIGS. 24 to 25 show the dose required, as predicted by the inverse-NN, for individuals from Data Set No. 3, Data Set No. 2, and Data Set No. 1, respectively.

The average, minimum, maximum, and standard deviation of the maximum bolus dose was required for each individual as calculated by the inverse-NN for each one of the 3 groups and for which the baseline aggregation will be kept at 20% for 24 hrs and 37 hrs are listed in Table 4.

TABLE 4

| | Data Set No. 3: Irish | Data Set No. 1: | Data Set No. 2: US Sick |
|---|---|---|---|
| | Sick Patients | Healthy Individuals | Patients |
| Dose (mg) NN-predicted to be required to | | | |
| achieve pattern No. 1 (keep 20% baseline | | | |
| aggregation level for 24 hrs) | | | |
| Average dose on patients in data set, mg | 19.3281 | 15.4936 | 19.5449 |
| Standard Deviation | 7.93066 | 1.96572 | 4.57417 |
| Maximum dose on patients in data set, mg | 32.4035 | 18.7409 | 26.6062 |
| Minimum dose on patients in data set, mg | 6.00026 | 10.6077 | 10.117 |
| Dose (mg) NN-predicted to be required to | | | |
| achieve pattern No. 2 (keep 20% baseline | | | |
| aggregation level for 36 hrs) | | | |
| Average dose on patients in data set, mg | 23.098 | 12.4137 | 21.8103 |
| Standard Deviation | 7.87113 | 3.25683 | 5.58016 |
| Maximum dose on patients in data set, mg | 33.1678 | 17.5387 | 30.8457 |
| Minimum dose on patients in data set, mg | 10.8899 | 4.28988 | 10.042 |

As mentioned before, among the two sets of patients, Data Set No. 3 is expected to have individuals which are sicker than individuals in Data Set No. 2, because they were scheduled to undergo angioplasty. Data set No. 1 comprised healthy volunteers that underwent clinical trials. Accordingly, it is expected that to maintain the same low levels of platelet aggregation, patients in data Set No. 3, No. 2, and No. 1 will require higher to the lower doses, respectively. The results of Table 4 indicate this is the case; i.e., higher doses are required for individuals in Data Set No. 3 than in Data Set No. 1. The differences become more dramatic if the time for which the 20% level of platelet aggregation is required needs to be extended. These results indicate that as the patient becomes sicker, not only does he or she require a higher dose in order to obtain a given effect, but also they become less capable of maintaining the response with the same dose.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Of course, variations of those preferred embodiments will become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method of predicting a drug dose necessary to achieve a desired drug effect using patient clinical characteristics, comprising:
inputting to a computer neural network a first data set comprising drug dose data, drug effect data, and patient characteristics data for a plurality of patients;
training the computer neural network on the first data set; and
using the computer neural network to predict a drug dose for a specific patient given a desired drug effect and patient characteristics of the specific patient.

2. The method of claim 1, wherein the drug dose data concerns the drug abciximab and the drug effect data concerns the inhibition of adenosine diphosphate (ADP)-induced platelet aggregation.

3. The method of claim 1, wherein the computer neural network is a backpropagation neural network.

4. The method of claim 1, wherein the computer neural network uses a steepest descent learning rule.

5. The method of claim 1, wherein training the computer neural network comprises establishing a relationship between the drug effect data and corresponding drug dose data and patient characteristics data.

6. The method of claim 1, wherein the computer neural network:
receives drug dose data and patient characteristics data;
predicts a drug effect based on the drug dose data and the patient characteristics data;
compares the predicted drug effect to received drug effect data; and
adjusts a weight in the computer neural network based on a difference between the predicted drug effect and the received drug effect data.

7. The method of claim 1, further comprising validating the computer neural network using a second data set comprising drug dose data, drug effect data, and patient characteristics data for a plurality of patients.

8. The method of claim 7, wherein validating the computer neural network comprises:
inputting to the computer neural network the drug dose data and the patient characteristics data; and
comparing a predicted drug effect to the drug effect data corresponding to the inputted drug dose data and patient characteristics data.

9. The method of claim 1, wherein the drug dose data is a drug dose versus time signature and the drug effect data is a drug effect versus time signature.

10. The method of claim 1, wherein the patient characteristics data includes data concerning at least one of ethnicity, age, gender, weight; stable angina, presence of diabetes, blood pressure, use of nitrates, cholesterol level, use of a statin, use of a beta blocker, use of a calcium blocker, use of a diuretic, smoking history, and previous myocardial infarctions.

11. The method of claim 10, wherein the patient characteristics data includes data concerning weight, smoking history, and previous myocardial infarctions.

12. A computer-readable medium having thereon computer-readable instructions for performing the steps comprising:
receiving a first data set comprising drug dose data, drug effect data, and patient characteristics data for a plurality of patients;
establishing a relationship between the drug effect data, the drug dose data, and the patient characteristics data in a neural network; and
predicting a drug dose for a specific patient given a desired drug effect and patient characteristics of the specific patient.

13. The method of claim 12, wherein the drug dose data concerns the drug abciximab and the drug effect data concerns the inhibition of adenosine diphosphate (ADP)-induced platelet aggregation.

14. The method of claim 12, wherein the neural network is a backpropagation neural network.

15. The method of claim 12, wherein the neural network uses a steepest descent learning rule.

16. The method of claim 12, wherein establishing the relationship includes:
predicting a drug effect based on the drug dose data and the patient characteristics data;
comparing the predicted drug effect to received drug effect data; and
adjusting a weight in the neural network based on a difference between the predicted drug effect and the received drug effect data.

17. The method of claim 12, wherein the drug dose data is a drug dose versus time signature and the drug effect data is a drug effect versus time signature.

18. The method of claim 12, wherein the patient characteristics data includes data concerning at least one of ethnicity, age, gender, weight, stable angina, presence of diabetes, blood pressure, use of a nitrate, cholesterol level, use of a statin, use of a beta blocker, use of a calcium blocker, use of a diuretic, smoking history, and previous myocardial infarctions.

19. The method of claim 18, wherein the patient characteristics data includes data concerning weight, smoking history, and previous myocardial infarctions.

20. A method of predicting a drug dose necessary to achieve a desired drug effect using patient clinical characteristics, comprising:
inputting to a first computer neural network a first data set comprising drug dose data, drug effect data, and patient characteristics data for a plurality of patients;
training the first computer neural network on the first data set;
using the first computer neural network to generate a second data set comprising drug dose data, drug effect data, and patient characteristics data for a plurality of hypothetical patients;
inputting to a second neural network the second data set;
training the second neural network on the second data set; and
using the second neural network to predict a drug dose for a specific patient given a desired drug effect and patient characteristics of the specific patient.

21. The method of claim 20, wherein the first computer neural network and the second computer neural network are backpropagation neural networks.

22. The method of claim 20, wherein the first computer neural network and the second computer neural network use a steepest descent learning rule.

23. The method of claim 20, wherein training the first computer neural network comprises establishing a relationship between the drug effect data and corresponding drug dose data and patient characteristics data.

24. The method of claim 20, wherein the first computer neural network:
receives drug dose data and patient characteristics data;
predicts a drug effect based on the drug dose data and the patient characteristics data;
compares the predicted drug effect to received drug effect data; and
adjusts a weight in the first computer neural network based on a difference between the predicted drug effect and the received drug effect data.

25. The method of claim 24, wherein the second computer neural network:
receives drug effect data and patient characteristics data;
predicts a drug dose based on the drug effect data and the patient characteristics data;
compares the predicted drug dose to received drug dose data; and
adjusts a weight in the second computer neural network based on a difference between the predicted drug dose and the received drug dose data.

26. The method of claim 20, wherein training the second computer neural network comprises establishing a relationship between the drug dose data and corresponding drug effect data and patient characteristics data.

27. The method of claim 20, further comprising validating the first computer neural network using a third data set comprising drug dose data, drug effect data, and patient characteristics data for a plurality of patients.

28. The method of claim 27, wherein validating the first computer neural network comprises:
inputting to the first computer neural network the drug dose data and the patient characteristics data; and
comparing a predicted drug effect to the drug effect data corresponding to the inputted drug dose data and patient characteristics data.

29. The method of claim 20, further comprising validating the second computer neural network using a third data set comprising drug dose data, drug effect data, and patient characteristics data for a plurality of patients.

30. The method of claim 29, wherein validating the second computer neural network comprises:
inputting to the second computer neural network the drug effect data and the patient characteristics data; and
comparing a predicted drug dose to the drug dose data corresponding to the inputted drug effect data and patient characteristics data.

31. The method of claim 20, wherein the drug dose data is a drug dose versus time signature and the drug effect data is a drug effect versus time signature.

32. The method of claim 20, wherein the patient characteristics data includes data concerning at least one of ethnicity, age, gender, weight, stable angina, presence of diabetes, blood pressure, use of a nitrate, cholesterol level, use of a statin, use of a beta blocker, use of a calcium blocker, use of a diuretic, smoking history, and previous myocardial infarctions.

33. The method of claim 32, wherein the patient characteristics data includes data concerning weight, smoking history, and previous myocardial infarctions.

34. The method of claim 20, wherein the drug dose data concerns the drug abciximab and the drug effect data concerns the inhibition of adenosine diphosphate (ADP)-induced platelet aggregation.

35. The method of claim 20, further comprising training the second computer neural network on a fourth data set comprising drug dose data, drug effect data, and patient characteristics data for a plurality of patients.

36. The method of claim 20, wherein using the second neural network to predict a drug dose comprises inputting the desired drug effect data and the patient characteristics and obtaining a predicted drug dose from the neural network that achieves the desired drug effect for the specific patient.

37. A computer-readable medium having thereon computer-readable instructions for performing the steps comprising:
receiving a first data set comprising drug dose data, drug effect data, and patient characteristics data for a plurality of patients;
establishing a relationship between the drug effect data, the drug dose data, and the patient characteristics data of the first data set in a first neural network;
generating a second data set comprising drug dose data, drug effect data, and patient characteristics data for a plurality of hypothetical patients;
establishing a relationship between the drug effect data, the drug dose data, and the patient characteristics data of the second data set in a second neural network; and
predicting a drug dose for a specific patient given a desired drug effect and patient characteristics of the specific patient using the second neural network.

38. The method of claim 37, wherein the first neural network and the second neural network are backpropagation neural networks.

39. The method of claim 37, wherein the first neural network and the second neural network use a steepest descent learning rule.

40. The method of claim 37, wherein establishing the relationship in the first neural network includes:
predicting a drug effect based on the drug dose data and the patient characteristics data;
comparing the predicted drug effect to received drug effect data; and
adjusting a weight in the first neural network based on a difference between the predicted drug effect and the received drug effect data.

41. The method of claim 37, wherein establishing the relationship in the second neural network includes:
receiving drug effect data and patient characteristics data;
predicting a drug dose based on the drug effect data and the patient characteristics data;
comparing the predicted drug dose to received drug dose data; and
adjusting a weight in the second neural network based on a difference between the predicted drug dose and the received drug dose data.

42. The method of claim 37, wherein the drug dose data is a drug dose versus time signature and the drug effect data is a drug effect versus time signature.

43. The method of claim 37, wherein the patient characteristics data includes data concerning at least one of ethnicity, age, gender, weight, stable angina, presence of diabetes, blood pressure, use of a nitrate, cholesterol level, use of a statin, use of a beta blocker, use of a calcium blocker, use of a diuretic, smoking history, and previous myocardial infarctions.

44. The method of claim 43, wherein the patient characteristics data includes data concerning weight, smoking history, and previous myocardial infarctions.

45. The method of claim 37, wherein the drug dose data concerns the drug abciximab and the drug effect data concerns the inhibition of adenosine diphosphate (ADP)-induced platelet aggregation.

46. The method of claim 37, wherein predicting a drug dose comprises receiving the desired drug effect data and the patient characteristics and outputting a predicted drug dose from the second neural network that achieves the desired drug effect for the specific patient.
